(19) United States
(12) Patent Application Publication
(10) Pub. No.: US 2024/0197646 A1
(43) Pub. Date: Jun. 20, 2024

(54) DRUG DELIVERY SYSTEM AND METHODS OF USING THE SAME

(71) Applicant: Axogen Corporation, Alachua, FL (US)

(72) Inventors: Kasra TAJDARAN, Alachua, FL (US); Justin DEUERLING, Alachua, FL (US); Angelo SCOPELIANOS, Alachua, FL (US); Xeve SILVER, Alachua, FL (US); Robert C. DILUCCIO, Alachua, FL (US); Nesreen ALSMADI, Alachua, FL (US)

(73) Assignee: Axogen Corporation, Alachua, FL (US)

(21) Appl. No.: 18/542,454

(22) Filed: Dec. 15, 2023

Related U.S. Application Data
(60) Provisional application No. 63/505,119, filed on May 31, 2023.
(60) Provisional application No. 63/476,268, filed on Dec. 20, 2022.

(51) Int. Cl. A61K 9/70; A61K 31/436; A61K 47/32; A61P 25/02

(52) U.S. Cl. CPC A61K 9/7007; A61K 31/436; A61K 47/32; A61P 25/02

(57) ABSTRACT

A method of preparing an implantable biomaterial film includes inputting a combination of a polymer and a neuro-regenerative agent or an immunosuppressive agent into an extruder. The method includes melting the polymer within the extruder. The method also includes extruding the combined polymer and the neuro-regenerative agent or the immunosuppressive agent to form the implantable biomaterial film.

### CROSS-REFERENCE TO RELATED APPLICATIONS

This patent application claims the benefit under 35 U.S.C. § 120 to U.S. Provisional Patent Application No. 63/476,268, filed on Dec. 20, 2022, and to U.S. Provisional Patent Application No. 63/505,119, filed on May 31, 2023, the entire contents of each of which are incorporated herein by reference.

### TECHNICAL FIELD

The present disclosure relates generally to the fields of tissue repair and medicine. More particularly, the present disclosure relates to biomaterials and drug delivery devices or platforms containing regenerative compounds, including neuro-regenerative compounds, as well as methods of making biomaterials and drug delivery devices, and methods of treatment using these biomaterials and drug delivery devices.

### BACKGROUND

Nerve damage, regardless of cause, may result in significant, and in some cases severe, disability and dysfunction. Neuropathic injury, in particular, can cause chronic pain, loss of sensation, loss of some or all muscle control, or other undesirable effects. Addressing the deleterious effects of peripheral nerve injury is a considerable challenge, particularly when there is a delay in nerve repair or when axons are required to reestablish connections with peripheral targets over large nerve defects or long distances. In such cases, the regenerating axons often do not have the required chemical and physiological cues to effectively regenerate and to reinnervate their end-target organs. For example, relatively long nerve gaps may experience a depletion of neurotrophic factors at the proximal nerve stump, and the concentration of neurotrophic factors may decline in a growth-supportive environment in the distal nerve stump.

Despite recent developments in surgical techniques, a limited number of patients with peripheral nerve injury recover full function. Therefore, it is desirable to develop clinically-applicable techniques for treating nerve injuries and restoring sensory and functional outcomes after nerve injuries. To promote effective restoration of function and sensation following nerve injury and repair, the intervention or the treatment should support axonal regeneration and/or increase the number of neurons regenerating their axons.

### SUMMARY

In accordance with the present disclosure, a biomaterial may include a regenerative agent, such as a neuro-regenerative agent, or an immunosuppressive agent. The biomaterial may be useful at the site of tissue injury and direct repair (e.g., direct nerve repair) or together with an implant (e.g., a nerve graft), may be attached to an implant (e.g., secured to a surface of an implant), or may be incorporated as part of an implant. In particular, a biomaterial may include FK506 incorporated into a film or sheet-shaped biomaterial that is suitable for implantation at or near an injured nerve. Alternatively, a biomaterial may include rapamycin incorporated into a film or sheet-shaped biomaterial. In another alternative, a biomaterial may include nimodipine incorporated into a film or sheet-shaped biomaterial. In this way, the biomaterial may be used to form a local drug delivery system for promoting the repair of injured tissue, for example, nerve tissue.

In one aspect, a method of preparing an implantable biomaterial film may include inputting a combination of a polymer and a neuro-regenerative agent or an immunosuppressive agent into an extruder. The method may include melting the polymer within the extruder. The method may also include extruding the combined polymer and the neuro-regenerative agent or the immunosuppressive agent to form the implantable biomaterial film.

In another aspect, a method of preparing an implantable biomaterial film may include inputting a combination of a polymer and FK506 into an extruder. The method may include melting the polymer. The method may include extruding the polymer and the FK506 using a film die to form an implantable biomaterial film. The method may also include cooling the implantable biomaterial film and collecting the implantable biomaterial film including the combined polymer and FK506.

In yet another aspect, an implant may comprise an extruded film, the extruded film comprising polydioxanone, alone or copolymerized with a second polymer, and FK506, rapamycin, or nimodipine.

Other objects, features, and advantages of the present disclosure will become apparent from the following detailed description. It should be understood, however, that the detailed description and the examples, while indicating exemplary embodiments of the present disclosure, are given by way of illustration only, since various changes and modifications within the spirit and scope of the invention will become apparent to those skilled in the art from this detailed description. Note that simply because a particular compound is ascribed to one generic formula does not mean that it cannot also belong to another generic formula.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass±10% of a specified amount or value. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more. As used herein, the terms “implantable,” “implantation,” and “implant” do not require placement within a subject such that the implant is under skin or other tissue. Rather the term “implant” additionally encompasses patches for placement on skin tissue, wraps for skin tissue, and devices for placement on or near mucous membranes or on a surface of other tissue.

Embodiments of this disclosure involve the use of a neuro-regenerative or an immunosuppressive agent. As used herein, the phrase “neuro-regenerative or immunosuppressive agents” refers to: one or more neuro-regenerative agents (e.g., nimodipine) and the absence of an immunosuppressive agent, the absence of one or more neuro-regenerative agents and the presence of an immunosuppressive agent, a single neuro-regenerative agent and a single immunosuppressive agent that are different from each other, the presence of a single agent that is both a neuro-regenerative agent and an immunosuppressive agent (e.g., FK506 or rapamycin), a plurality of neuro-regenerative agents and a plurality of immunosuppressive agents, a single neuro-regenerative agent and a plurality of immunosuppressive agents, or a plurality of neuro-regenerative agents and a single immunosuppressive agent, regardless of whether the phrase “neuro-regenerative or immunosuppressive agent” is presented in singular or plural form, or shortened to the term “agent(s)” or “agent”. Further, although neuro-regenerative agents for nerve repair are described herein, it is contemplated that regenerative agents suitable for use with tissues other than nerves may be used.

Both the foregoing general description and the following detailed description are exemplary and explanatory only and are not restrictive of the features, as claimed. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. The terms “film” and “sheet” are considered interchangeable within and refer to thin pieces of material (e.g., a thickness of less than 200 μm) of any shape that have a thickness that is less than their length and width. Additionally, the term “exemplary” is used herein in the sense of “example,” rather than “ideal.” In addition, the term “between” used in describing ranges of values is intended to include the minimum and maximum values described herein.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of exemplary embodiments presented herein.

FIG. 1 shows a schematic diagram of an exemplary process for incorporating one or more neuro-regenerative or immunosuppressive agents in a polymer, according to aspects of the present disclosure.

FIG. 2 shows a schematic diagram of an exemplary process for producing a film containing one or more neuro-regenerative or immunosuppressive agents, according to aspects of the present disclosure.

FIG. 3 shows a flowchart of an exemplary process for incorporating one or more neuro-regenerative or immunosuppressive agents in a polymer, according to aspects of the present disclosure.

FIG. 4 shows a flowchart of an exemplary process for producing a film containing one or more neuro-regenerative or immunosuppressive agents, according to aspects of the present disclosure.

FIG. 5A shows an exemplary nerve wrap implant formed with a biomaterial film including one or more neuro-regenerative or immunosuppressive agents, according to aspects of the present disclosure.

FIG. 5B shows an exemplary nerve connector implant used with a biomaterial film including one or more neuro-regenerative or immunosuppressive agents, according to aspects of the present disclosure.

FIG. 5C shows an exemplary rolled nerve wrap implant used with a biomaterial film including one or more neuro-regenerative or immunosuppressive agents, according to aspects of the present disclosure.

FIG. 5D shows a tissue-derived implant used with a biomaterial film including one or more neuro-regenerative or immunosuppressive agents, according to aspects of the present disclosure.

FIG. 6 is a graph depicting the release of an exemplary neuro-regenerative or immunosuppressive agent from a film, according to aspects of the present disclosure.

FIG. 7 is a chart depicting neurite outgrowth in an exemplary in vitro use of a film having one or more neuro-regenerative or immunosuppressive agents incorporated in the film, and exemplary comparative examples, according to aspects of the present disclosure.

FIGS. 8A-8D are fluoroscopy images corresponding to the exemplary in vitro use of a film having one or more neuro-regenerative or immunosuppressive agents incorporated in the film, and exemplary comparative examples, according to aspects of the present disclosure.

### DETAILED DESCRIPTION

The biomaterials that serve as a local drug delivery device of the present disclosure may incorporate one or more neuro-regenerative or immunosuppressive agents in a polymeric material. The biomaterial may be useful with a nerve implant or as a nerve implant (e.g., a nerve wrap, nerve connector, pre-rolled nerve wrap, etc.), or may be implanted separately (e.g., at or near the site of an injury or other location at which a nerve implant has been or will be implanted) to form a local drug delivery device. The biomaterial may have a suitable shape, such a continuous sheet, a rolled sheet, a perforated sheet, or a plurality of separate (e.g., discrete) sheets. The one or more regenerative, e.g., neuro-regenerative, or immunosuppressive agents may be distributed throughout the biomaterial or implant. The biomaterial may be distributed throughout a tissue implant, such as a nerve implant, may be localized in one or more surfaces or regions of a tissue implant, or may form a part or the entirety of the structure of the tissue implant. The biomaterials of the present disclosure may promote tissue regeneration, e.g., nerve regeneration, which, in some aspects, may in turn improve nerve regeneration outcomes. Exemplary biomaterials, related methods for their preparation, and related methods of treatment using biomaterials, are described in detail below. While the local drug delivery systems, biomaterials, implants, and methods herein are discussed with respect to use at a nerve site, the local drug delivery systems, biomaterials, implants, and method may be applied to other types of tissues and other locations in a subject.

The biomaterial may include a polymer that is compatible for use in conjunction with a nerve implant. The polymer may be compatible with one or more neuro-regenerative or immunosuppressive agents. The polymer may be biodegradable following implantation in a human or non-human animal. The polymer may comprise homopolymers, copolymers, and/or polymeric blends including one or more of the following monomers: glycolide, lactide (d,l-lactide or l-lactide), caprolactone, dioxanone, trimethylene carbonate, monomers of cellulose derivatives, and monomers that polymerize to form polyesters. The polymer may include polydioxanone (PDS), polycaprolactone (PCL), polytrimethylene carbonate, polyglycolide (PGL), poly-3-hydroxybutyrate (PHB), poly(3-hydroxybutyrate-co-3-hydroxyvalerate) (PHBV), poly(propylene carbonate) (PPC), poly(butylene succinate) (PBS), poly(propylene fumarate) (PPF). The polymer may be formulated by two or more of these monomers. The polymer may be a copolymer that includes dioxanone co-polymerized with trimethylene carbonate, 1-lactide, or caprolactone.

The polymer may be co-polymerized with lactide and/or glycolide. The polymer may be a copolymer that includes dioxanone co-polymerized with lactide and/or glycolide such that polymerized dioxanone forms a majority of the polymer, by weight. The polymer may include polylactic acid (PLA) or poly(lactic-co-glycolic acid) (PLGA). However, in some aspects, the polymer may be free of one or both of PLA and PLGA. The polymer (e.g., PDS copolymerized with PLA and/or PLGA) may be surface treated. For example, the polymer may be surface treated with a polyethylene glycol (PEG), such as PEG 1000.

The one or more neuro-regenerative or immunosuppressive agents may include an immunophilin ligand. The one or more neuro-regenerative or immunosuppressive agents may include FK506 (tacrolimus), rapamycin (rapamune, or sirolimus), cyclosporine A, or nimodipine. The one or more neuro-regenerative or immunosuppressive agents may be hydrophobic, with a melting point below about 200 degrees Celsius, below about 120 degrees Celsius, below about 130 degrees Celsius, or below about 110 degrees Celsius.

The one or more neuro-regenerative or immunosuppressive agents may be mixed with the polymer at any suitable concentration, as described below. The biomaterial may be manufactured by creating a so-called “masterbatch”, also referred to herein as a “stock” batch of biomaterial, containing polymer and a desired concentration of one or more neuro-regenerative or immunosuppressive agents (e.g., about 1%, about 2%, about 3%, about 4%, about 5%, about 6%, or more, by weight of a neuro-regenerative or immunosuppressive agent, such as FK506, rapamycin, or nimodipine). Alternatively, the biomaterial may be manufactured by combining a polymer with a neuro-regenerative or immunosuppressive agent without the use of a stock batch (e.g., a biomaterial formed by combining one or more neuro-regenerative or immunosuppressive agents with a polymer that is free of these agents and performing a single extrusion for forming a sheet-shaped biomaterial). The stock batch may be useful to provide tailoring for specific uses for local drug delivery, such that the stock batch of material may be employed in subsequent processing to produce one or more biomaterials having one of multiple possible final forms with a suitable concentration of agent, as described below. If desired, the different biomaterials created may be used as the building blocks to create different local drug delivery systems, depending on the need.

The biomaterial may be provided in a suitable form factor whether the biomaterial is an intermediate product (e.g., when the biomaterial is part of a stock batch) or a final form (e.g., a product that is intended for inclusion in or attachment to an implant, e.g., a nerve implant, or use independent of a nerve implant). The biomaterial may be formed as one or more pellets (e.g., a stock batch of biomaterial) or one or more films or sheets (e.g., a biomaterial for implantation). The film(s) or sheet(s) may have a thickness, width, and/or length that varies and can be selected according to the intended use or desired properties (e.g., desired handling properties).

As discussed above, the biomaterial may be provided in the form of one or more sheets. The sheet may be a nonwoven sheet. The biomaterial may be formed via extrusion of a polymer that is shaped into one or more sheets due to the shape of a die through which the biomaterial is extruded. The biomaterial sheet may include a constant or relatively constant concentration of the one or more neuro-regenerative or immunosuppressive agents along its width or length.

In some aspects, the biomaterial may be integrated with a nerve implant (e.g., one or more portions of the implant may be formed of or may incorporate the biomaterial), the biomaterial may be attached to the implant (e.g., the biomaterial may be wrapped around or otherwise connected to an interior or exterior portion of the implant), or the biomaterial may be implanted at the same area as a nerve implant. The biomaterial may have any suitable form factor, whether integrated with an implant, attached to an implant, or implanted in the same area as an implant. The nerve implant may be provided for various uses, including use as a nerve connector, nerve wrap, nerve graft, nerve protector, etc. In particular, the biomaterial may be provided as part of, or for use with, implants such as those described in U.S. patent application Ser. No. 15/344,908, filed on Nov. 7, 2016, which issued as U.S. Pat. No. 10,835,253; U.S. patent application Ser. No. 15/252,917, filed on Aug. 31, 2016, which issued as U.S. Pat. No. 10,945,737; U.S. patent application Ser. No. 15/900,971, filed on Feb. 21, 2018, which issued as U.S. Pat. No. 10,813,643; U.S. Ser. No. 16/381,860, filed on Apr. 11, 2019, which issued as U.S. Pat. No. 11,166,800; U.S. patent application Ser. No. 16/192,261, filed on Nov. 15, 2018, which issued as U.S. Pat. No. 11,477,558; U.S. patent application Ser. No. 14/036,405, filed on Sep. 25, 2013, which issued as U.S. Pat. No. 9,629,997; U.S. application Ser. No. 16/898,224, filed on Jun. 10, 2020; or U.S. patent application Ser. No. 17/451,489, filed on Oct. 20, 2021.

While the implant may be a nerve implant, the implant may be an implant other than a nerve implant. Any of the biomaterials discussed herein may be useful with implants other than nerve implants. In some aspects, the biomaterials and/or implants may be useful for vascular implantation, implantation into or placement on the surface of skin (e.g., as a topical, a transdermal patch, etc.), skeletal implantation, spinal implantation, urological implantation, tendon implantation, muscular implantation, and/or others. The one or more neuro-regenerative or immunosuppressive agents may, when incorporated within an implant other than a nerve implant, be an immunosuppressive agent. These agents may have other properties that are beneficial to the location of implantation, and/or may be provided with additional compounds that provide beneficial properties. For example, an implant intended for vascular implantation may include an antiproliferative agent that inhibits neointimal hyperplasia, such as paclitaxel, in addition to one or more immunosuppressive agents.

When the implant is a nerve implant, the nerve implant may be formed with tissue, e.g., nerve graft tissue, to which a film-shaped biomaterial is connected. For example, nerve tissue may have one or more film-shaped biomaterials applied to an exterior (e.g., as a wrap) of the nerve tissue. Nerve graft tissue suitable for processing according to the methods herein may be natural or synthetic. For example, the tissue may be soft biological tissue obtained from an animal, such as a mammal, including a human or a non-human mammal, or a non-mammal, including a fish, amphibian, or insect. The tissue may be allogeneic or xenogeneic to a subject into which the graft is implanted. The tissue may be nerve tissue, including, for example, peripheral nerve tissue or central nervous system tissue. Other types of tissue suitable for the present disclosure include, but are not limited to, epithelial tissue, connective tissue, muscular tissue, capillary tissue, dermal tissue, skeletal tissue, smooth muscle tissue, cardiac tissue, and adipose tissue. As mentioned above, the soft biological tissue may be mammalian tissue, including human tissue and tissue of other primates, rodent tissue, equine tissue, canine tissue, rabbit tissue, porcine tissue, or ovine tissue. In addition, the tissue may be non-mammalian tissue, selected from piscine, amphibian, or insect tissue. The tissue may be a synthetic tissue, such as, but not limited to, laboratory-grown or 3D-printed tissue. According to some examples, the tissue is nerve tissue obtained from an animal, such as a human or a non-human mammal. The tissue may be obtained and/or treated as disclosed in U.S. patent application Ser. No. 17/411,718, entitled “Nerve Grafts and Methods of Preparation Thereof,” filed on Aug. 25, 2021, the entirety of which is incorporated by reference. In at least some embodiments, an exemplary tissue may be a processed human nerve allograph, such as an Avance® Nerve Graft from Axogen, Inc. (Alachua, FL, US).

Although embodiments of the disclosure are described in relation to biomaterials useful for nerve injury, and in particular, to biomaterials films that form nerve implants or that are used with nerve implants for peripheral nerve injury, it is contemplated that other types of tissue, including any of the materials described above, may be used in the methods and biomaterial films described herein.

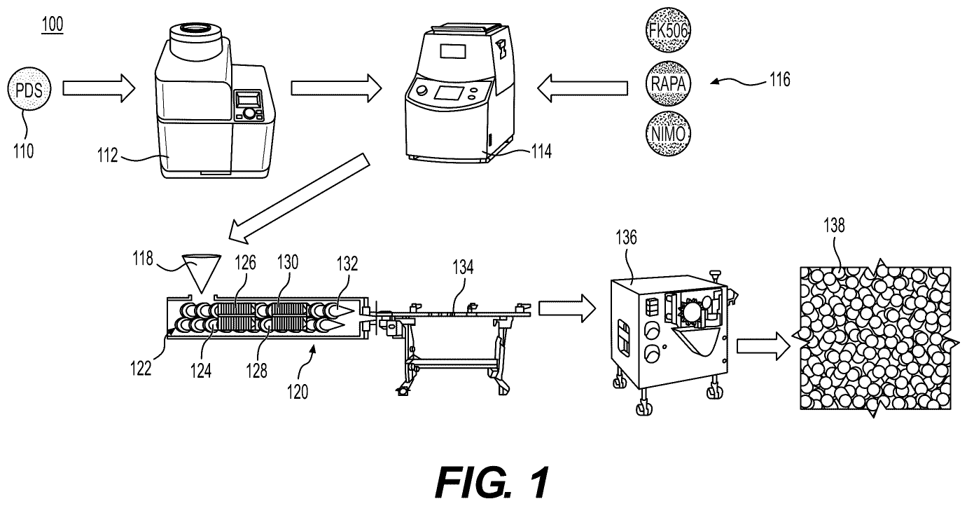

FIG. 1 illustrates a diagram of an exemplary process 100 for producing a biomaterial, such as biomaterial 138 (particles such as spheres, pellets, etc., being shown in FIG. 1), which may include a polymer and an immunosuppressive or neuro-regenerative agent. Biomaterial 138 may be suitable for processing and subsequent implantation in a human or non-human animal. For example, biomaterial 138 may be suitable for further processing and subsequent use with a nerve implant and/or for implantation in a human or non-human animal. A biomaterial 138 may also be formed in process 100. Biomaterial 138 may form a “stock batch” of biomaterial, as described in detail below.

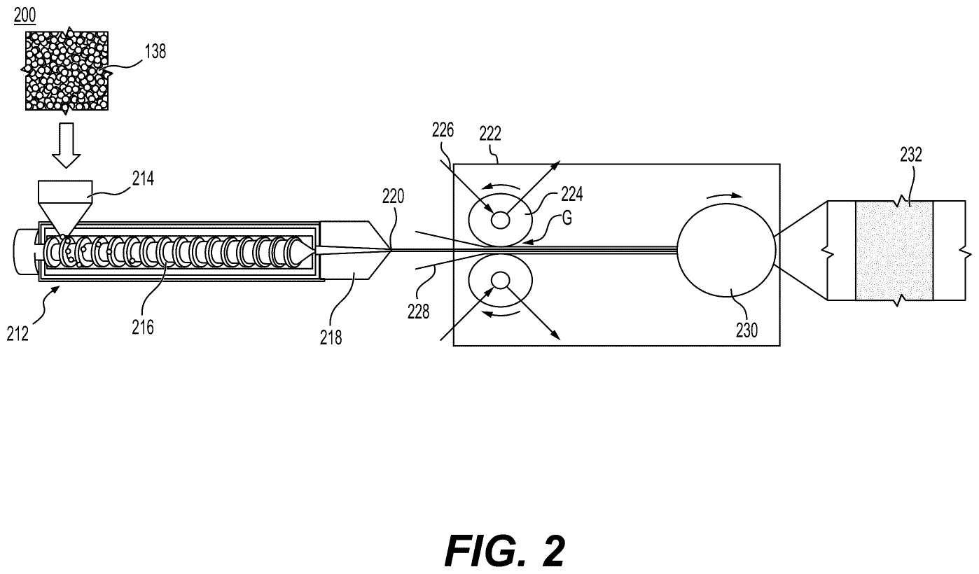

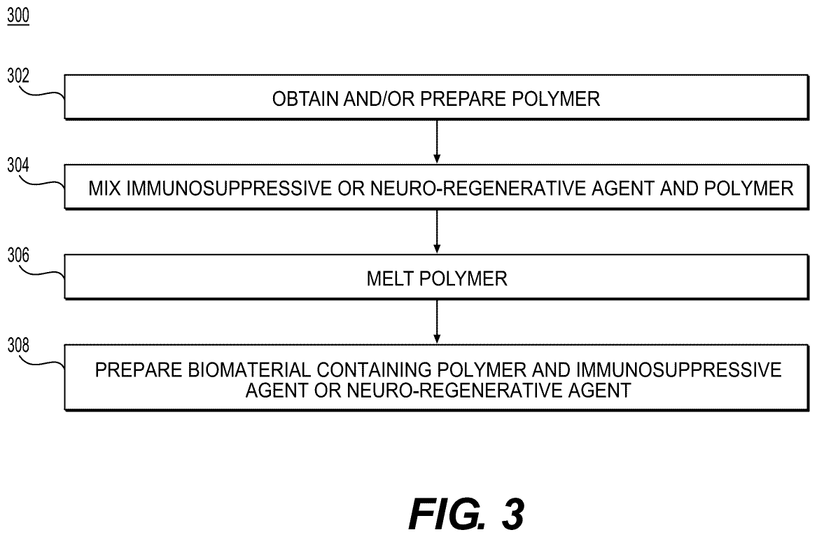

FIG. 3 illustrates a flowchart of an exemplary process 300 for producing a biomaterial that includes a polymer and one or more immunosuppressive or neuro-regenerative agents, such as FK506, rapamycin, or nimodipine. While process 300 is described in conjunction with process 100 and FIGS. 1 and 2 below, as understood, process 300 may include fewer steps, additional steps, and/or different steps as compared to process 100. Additionally, process 300 may include fewer steps, additional steps, and/or different steps as compared to each block (e.g., steps 302, 304, 306, and 308) illustrated in FIG. 3, or the specific order of the steps may be different. In some aspects, one or more of steps 302, 304, 306, and 308 may be repeated one or more times.

In a step 302 (FIG. 3), a polymer 110 (FIG. 1) may be obtained. Polymer 110 may include a polyester. Polymer 110 may include polydioxanone (PDS), polycaprolactone (PCL), polyglycolide (PGL), poly-3-hydroxybutyrate (PHB), poly(3-hydroxybutyrate-co-3-hydroxyvalerate) (PHBV), poly(propylene carbonate) (PPC), poly(butylene succinate) (PBS) and poly(propylene fumarate) (PPF). Polymer 110 may include poly(lactic acid) (PLA) or poly(lactic-co-glycolic acid) (PLGA).

However, in at least some embodiments, polymer 110 may be free of both PLA and PLGA. Forming polymer 110 free of both PLA and PLGA may allow biomaterial 138 to avoid the generation of acid at the site of implantation that may occur when PLA or PLGA degrades following implantation.

In at least some examples, polymer 110 may include dioxanone co-polymerized with, for example, trimethylene carbonate, l-lactide, d,l-lactide, glycolide, or caprolactone. In particular, polymer 110 may include a PDS/poly(trimethylene carbonate) random copolymer, a PDS/poly(trimethylene carbonate) block copolymer, a PDS/poly(l-lactide) random copolymer, a PDS/poly(l-lactide) block copolymer, a PDS/poly(caprolactone) random copolymer, or a PDS/poly(caprolactone) block copolymer. In some examples, polymer 110 may include a PDS/poly(glycolide)/poly(l-lactide) random copolymer, a PDS/poly(glycolide)/poly(l-lactide) block copolymer, a PDS/poly(d,l-lactide) random copolymer, or a PDS/poly(d,l-lactide) block copolymer. Use of one or more random copolymers may reduce the crystallinity of the PDS material, thereby making the film material softer.

In some aspects, inclusion of a softer or more drapeable material may inhibit kinking during use. For example, if used to wrap around a nerve, kinking may be inhibited, which may inhibit nerve compression. Softness may be controlled (e.g., increased) by surface treating the polymer. For example, polyethylene glycol (PEG) may be used to surface treat polymer 110, for example, PDS/poly(glycolide)/poly(l-lactide) or PDS/poly(d,l-lactide). The molecular weight of the PEG may be about 1,000 daltons to about 10,000 daltons.

Introduction of one or more copolymers may allow the polymer film to degrade faster and release an incorporated drug faster during use. For example, introduction of one or more copolymers may cause the polymer to degrade within about 30 days, e.g., from about 5 to about 45 days, from about 10 to about 35 days, from about 20 to about 30 days, or from about 5 to about 30 days. In one aspect, a PDS/poly(glycolide)/poly(l-lactide) or PDS/poly(d,l-lactide) copolymer may have a degradation rate of about 60 days, or less, about 45 days, or less, or about 30 days, or less. This degradation rate may be increased (i.e., faster degradation) based on addition of PEG to increase hydrophilicity of the copolymer, resulting in increased degradation via hydrolysis. A faster degradation rate may also affect the release profile of any drugs incorporated into the copolymer. By adjusting the ratios of the components of polymer 110, the molecular weights of the components, and/or the use of surface treatments, is may be possible to alter the physical properties and hydrophilicity of the copolymer.

In some aspects, polymer 110 includes PDS and one of the above-described copolymers (e.g., poly(trimethylene carbonate), poly(l-lactide), or poly(caprolactone)), as a random copolymer, in a molecular weight ratio of about 50/50 (PDS:copolymer), about 60/40 (PDS:copolymer), about 70/30 (PDS:copolymer), about 80/20 (PDS:copolymer), or about 90/10 (PDS:copolymer). In some aspects, polymer 110 includes 0 to 50% random copolymer by molecular weight, or up to about 50% random copolymer, by molecular weight. In some aspects, the quantity of PDS in polymer 110, polymer 110 including a randomly-copolymerized copolymer, may be about 40% to about 90% PDS, or about 50% to about 80% PDS, by molecular weight.

In other aspects, polymer 110 includes PDS and one of the above-described copolymers (e.g., poly(trimethylene carbonate), poly(l-lactide), or poly(caprolactone)), as a block copolymer, in a ratio of about 50/50 (PDS:copolymer), about 60/40 (PDS:copolymer), about 70/30 (PDS:copolymer), about 80/20 (PDS:copolymer), or about 90/10 (PDS:copolymer), by molecular weight. In some aspects, the amount of PDS relative to the amount of a copolymer may be reduced for block copolymers as compared to the amount of PDS relative to the amount of a random copolymer. Thus, polymer 110, in some embodiments, contains PDS and a block copolymer such as poly(trimethylene carbonate), poly(l-lactide), or poly(caprolactone), in a ratio of about 45/55 (PDS:copolymer), about 55/45 (PDS:copolymer), about 65/35 (PDS:copolymer), about 75/25 (PDS:copolymer), or about 85/15 (PDS:copolymer), by molecular weight. In some aspects, polymer 110 includes 0 to 50% block copolymer by molecular weight, or up to about 50% block copolymer, by molecular weight. In some aspects, the quantity of PDS in polymer 110, polymer 110 including a block copolymer, may be about 50% to about 80% PDS, about 45% to about 85% PDS, about 55% to about 75% PDS, or about 60% to about 70% PDS, by molecular weight.

In further aspects, polymer 110 includes PDS, poly(glycolide), and poly(l-lactide) as block or random copolymers. The amount of PDS in the PDS/poly(glycolide)/poly(l-lactide) may be about 55% to about 90% as measured by molecular weight. The amount of poly(glycolide) in the PDS/poly(glycolide)/poly(l-lactide) may be about 5% to about 20% as measured by molecular weight. The amount of poly(l-lactide) in the PDS/poly(glycolide)/poly(l-lactide) may be about 2% to about 10% as measured by molecular weight. In one example, polymer 110 includes about 85% PDS, about 10% poly(glycolide), and about 5% poly(l-lactide). In one example, polymer 110 may include about 85% PDS, about 10% poly(glycolide), and about 5% poly(l-lactide). Any of the above-described PDS/poly(glycolide)/poly(l-lactide) copolymers may be surface treated with PEG having a molecular weight of 1,000 daltons to about 10,000 daltons.

In further aspects, polymer 110 includes PDS and poly(d,l-lactide) as a block or random copolymer. The amount of PDS in the PDS/poly(d,l-lactide) may be about 60% to about 90% as measured by molecular weight. The amount of poly(d,l-lactide) in the PDS/poly(d,l-lactide) may be about 10% to about 40% as measured by molecular weight. In one example, polymer 110 may include about 80% PDS and about 20% poly(d,l-lactide). Any of the above-described PDS/poly(d,l-lactide) copolymers may be surface treated with PEG having a molecular weight of 1,000 daltons to about 10,000 daltons.

If desired, the total mass of the PDS/poly(glycolide)/poly(l-lactide) or the PDS/poly(d,l-lactide) may be about 8,000 daltons to about 240,000 daltons, or about 10,000 daltons to about 100,000 daltons. The molecular weight may be modified by selecting from the above-described ratios or otherwise modifying the ratio of copolymers.

When polymer 110 contains poly(lactide) or poly(glycolide), a coating may be employed. In particular, a coating may be reduced to reduce acidity that occurs during degradation of the polymer following implantation. Exemplary coatings include magnesium carbonate, sodium carbonate, or other basic salts. An amount of the basic salt may be about 0.5% to about 10% by molecular weight. In particular, the amount of basic salt may be about 1% by molecular weight.

The obtained polymer 110 may be in any suitable form, such as powder, flakes, filaments, particles (spheres, pellets, etc.), among others. In some aspects, polymer 110 may be obtained in a form other than powder. When polymer 110 is in a form other than a powder, step 302 may include forming a powder by grinding particles, filaments, pellets, or another structure of polymer 110 to obtain a powder of polymer 110 with grains and/or particles that have a suitable size to facilitate homogenous mixing of polymer 110 and immunosuppressive or neuro-regenerative agent 116. Polymer 110 may be milled to a powdered form using, for example, a centrifugal mill 112. A process of milling polymer 110 may include obtaining powdered polymer with centrifugal mill 112 and drying the powder with a polymer dryer. An exemplary polymer dyer is a nitrogen-gas dryer. When drying is performed, a dryer may be used to dry milled polymer 110 for a suitable period of time, such as 1 hour, 2 hours, 4 hours, 6 hours, 12 hours, 24 hours, or more.

If desired, step 302 may include synthesizing polymer 110. For example, when polymer 110 is PDS, synthesis of polymer 110 may include a ring-opening polymerization reaction of p-dioxanone. The synthesis of polymer 110 may include causing a copolymerization reaction for polymer 110, including either random copolymerization or block copolymerization. Whether obtained as a powder, milled to a powder, and/or synthesized during step 302, polymer 110 may be a substantially pure powder that is suitable for mixing with the one or more immunosuppressive or neuro-regenerative agents, which may also be in a powdered form.

In a step 304 (FIG. 3), the one or more immunosuppressive or neuro-regenerative agents 116 (FIG. 1) and polymer 110 may be mixed together. Mixing may include physically blending agent(s) 116 and polymer 110. For example, agent(s) 116 and polymer 110, one or both of which are present in powdered form, may be thoroughly mixed with a mixer 114 together to produce uniform, homogenously-mixed blend of pellets having a desired ratio of agent(s) 116 to polymer 110. Mixing may be performed, for example, with a dual asymmetric centrifuge mixer. Polymer 110 and agent(s) 116 may be separately weighed and provided to mixer 114 to arrive at a homogenously-mixed powder containing the desired ratio of polymer 110 to agent(s) 116.

While agent(s) 116 and polymer 110 may be mixed when both are in a solid state, one or both of agent(s) 116 and polymer 110 may be in a liquid form during mixing. For example, agent(s) 116 and polymer 110 may be heated, dissolved in a solvent, etc., to create a liquid form suitable for mixing with another liquid. In embodiments where agent(s) 116 and polymer 110 are mixed while in a liquid form, melting agent(s) 116 and polymer 110 in step 306, described below, may be omitted.

Agent(s) 116 and polymer 110 may be provided to an extruder 120 after being mixed with mixer 114. For example, extruder 120 may include a hopper or feeder 118 configured to receive the mixed agent(s) and polymer 110.

In some embodiments, agent(s) 116 and polymer 110 may be provided separately to a system configured to both: mix agent(s) 116 and polymer 110, and to melt polymer 110 (as described below with respect to step 306 of method 300). For example, extruder 120 may include a plurality of feeding devices, such as a plurality of feeders 118, for separately receiving agent(s) 116 and polymer 110 in a solid form (e.g., as powders). When plural hoppers or other feeding devices 118 are present, these devices may be configured to compensate for changes in the agent(s) 116 and polymer 110 supplied to extruder 120, such as reductions in weight as material is depleted, in the example of gravity-fed feeding devices.

Extruder 120 may be configured to supply metered quantities of agent(s) 116 and metered quantities of polymer 110, whether agent(s) 116 and polymer 110 were mixed with mixer 114 or mixed via plural hoppers or feeders 118, to a mixing section 124. Feeder 118 may be configured to feed agent(s) 116 and polymer 110 in a controlled manner in which agent(s) 116 and polymer 110 are drawn to the interior of extruder 120.

Whether agent(s) 116 and polymer 110 are mixed before being supplied to extruder 120 or are mixed by extruder 120 itself, agent(s) 116 and polymer 110 may be metered such that the ratio of agent(s) 116 to polymer 110 is precisely controlled. For example, a concentration of agent 116 may be within a range of about 0.5% by weight to about 30% by weight of the biomaterial, within a range of about 1% by weight to about 20% by weight, or within a range of about 2% by weight to about 10% by weight. In particular, the concentration of agent 116 may be about 1% by weight, about 2% by weight, about 3% by weight, about 4% by weight, about 10% by weight, or about 20% by weight. The concentration of agent(s) 116 in the biomaterial 138 formed from the material output from extruder 120 may be substantially the same as the concentration of agent(s) 116 received by extruder 120, or the same as the concentration of agent(s) 116 at any point within extruder 120. Regardless, the concentration of agent(s) 116 within the extruded product may be any of the above-described concentrations.

A step 306 (FIG. 3) of process 300 may include melting polymer 110 (FIG. 1) received with extruder 120. This may include supplying the agent 116 and polymer 110 to a hopper 118 or other input connected to extruder 120. For example, hopper 118 may be a vibratory feeder that receives pre-mixed agent(s) 116 and polymer 110. In some aspects, polymer 110 may be obtained in a liquid form (e.g., a polymer 110 in a form that solidifies in the presence of air and/or a catalyst, or by heating polymer 110 before introducing polymer 110 into extruder 120). Thus, step 306 may be optional, or may be performed to maintain polymer 110 in a liquid state.

Extruder 120 may be a single-screw or twin-screw extruder that includes an extruder screw 122, screw 122 having one or more of a conveying section 124, a kneading section 126, a conveying section 128, a shearing section 130, and a metering section 132. Extruder 120 may be a twin-screw extruder that includes a pair of co-rotating or counter-rotating screws 122. Screw 122 may be sized appropriately for producing biomaterial 138 with or without additional processing. For example, screw 122 may have an outer diameter within a range of about 9 mm to about 36 mm, the outer diameter being a maximum diameter of screw 122. In particular, screw 122 may have a maximum outer diameter of about 18 mm.

Conveying section 124 of extruder screw 122 may include threading sized to receive agent(s) 116 and polymer 110 and convey both materials downstream. Additionally, conveying section 124 may include a zone that includes threading having a reduced thread pitch (threading that is spaced closer together) to generate heat to begin softening polymer 110. Kneading section 126 may receive agent(s) 116 and polymer 110. Threads of kneading section 126 may have a geometry suitable for generating heat, by friction, to melt polymer 110. The heat generated with kneading section 126 during step 306 may be sufficient to soften and at least partially melt polymer 110 without adversely impacting the effectiveness of agent(s) 116 due to overheating. This temperature may be within a range of about 40 degrees Celsius to about 200 degrees Celsius, for example, or about 80 degrees Celsius to about 150 degrees Celsius. The temperature generated with extruder 120 may be selected based on the softening and/or melting temperatures of agent 116 and polymer 110, or to avoid a temperature at which agent(s) 116 may be damaged or deactivated, e.g., denatured.

A second conveying section 128 may receive heated material from kneading section 126 and supply this material to a shearing section 130, which includes shearing or kneading threads configured to further increase the temperature of agent 116 and polymer 110. The temperature generated with shearing section 130 may be a maximum temperature generated with extruder 120. This maximum temperature within extruder 120 may be higher than a melting temperature of agent(s) 116 and higher than a melting temperature of polymer 110. In examples where agent 116 is FK506 and polymer 110 is PDS, shearing section 130 may be configured to generate a temperature within a range of about 110 degrees Celsius to about 155 degrees Celsius. In examples where agent 116 includes FK506, shearing section 130 may raise the temperature of agent(s) 116 and polymer 110 from a temperature of about 100 degrees Celsius to a temperature in a range of about 120 degrees Celsius to about 155 degrees Celsius. In particular, shearing section 130 may raise the temperature of agent 116 and polymer 110 to about 135 degrees Celsius. This temperature may be sufficient to ensure polymer 110 is in a liquid state before agent(s) 116 and polymer 110 are received by a die at a downstream end of extruder 120.

In examples where agent 116 includes rapamycin and polymer 110 is PDS, shearing section 130 may be configured to generate a temperature within a range of about 100 degrees Celsius to about 250 degrees Celsius. For example, shearing section 130 may raise the temperature of agent(s) 116 and polymer 110 to a temperature in a range of about 150 degrees Celsius to about 250 degrees Celsius. In particular, shearing section 130 may raise the temperature of agent 116 and polymer 110 to a temperature in a range of about 175 degrees Celsius to about 225 degrees Celsius. Shearing section 130 may raise the temperature of agent(s) 116 and polymer 110 to a temperature above about 185 degrees Celsius.

In examples where agent 116 includes nimodipine and polymer 110 is PDS, shearing section 130 may be configured to generate a temperature within a range of about 100 degrees Celsius to about 200 degrees Celsius. For example, shearing section 130 may raise the temperature of agent(s) 116 and polymer 110 to a temperature in a range of about 120 degrees Celsius to about 180 degrees Celsius. In particular, shearing section 130 may raise the temperature of agent 116 and polymer 110 to a temperature in a range of about 130 degrees Celsius to about 170 degrees Celsius. Shearing section 130 may raise the temperature of agent(s) 116 and polymer 110 to a temperature above about 125 degrees Celsius.

While the heat generated by extruder 120 may be generated entirely by friction caused by the rotation of extruder screw(s) 122, one or more heaters may be secured to extruder 120 to assist in the generation of a desired amount of heat and maintenance of a desired temperature at one or more locations within extruder 120. These heaters may be placed along one or more positions of the barrel of extruder 120, and may partially or entirely surround kneading section 126, shearing section 130, and/or any other sections of extruder screw 122. A temperature may be monitored at one or more locations along the length of extruder 120. For example, one or more temperature sensors may be positioned to detect a temperature within extruder 120. A control system, in communication with these temperature sensors, may control the heaters on the barrel of extruder 120 to maintain a desired temperature. Sensor(s) may be provided to monitor and control one or more other aspects of the extrusion process, such as torque applied to screw 122, pressures within extruder 120, etc.

A step 308 (FIG. 3) may include extruding agent(s) 116 (FIG. 1) and polymer 110 with extruder 120, for example. Melted and mixed agent(s) 116 and polymer 110 may be conveyed and metered via metering section 132 downstream of shearing section 130. Metering section 132 may supply homogenized agent 116 and polymer 110 to an extruder die having an opening with a diameter of about 0.5 mm to about 8 mm, or other diameters suitable for extruding rods having a final diameter of about 50 μm to about 3 mm or larger (e.g., with use of a puller to draw down the diameter of the rods). In other aspects, the extruder die may have an opening with a diameter of about 50 μm to about 200 μm for producing extruded rods having a final diameter of about 50 μm to about 200 μm. In particular, the extruder die may have an opening with about a 1 mm diameter.

A conveyor 134 may receive material, in the form of fibers or rods, output from the die of extruder 120. Conveyor 134 may allow the material output from extruder 120 to cool. Cooling may be performed passively or actively (e.g., by supplying coolant to reduce a temperature of conveyor 134, or by directing cooling air towards a surface of conveyor 134).

The extruded material, which includes agent(s) 116 and polymer 110, may be received by a pelletizer or other shaping device 136 downstream of conveyor 134. While metering section 132 may be formed by a downstream portion of extruder screw(s) 122, metering section 132 may include a metering pump (e.g., a gear pump) configured to push a precisely-metered quantity of combined agent(s) 116 and polymer 110, to an output of extruder 120.

Metering section 132 may supply combined agent(s) 116 and polymer 110, at a desired rate, to shaping device 136 via conveyor 134. Shaping device 136 may be any suitable device or plurality of devices configured to modify or otherwise control the shape of the product extruded with extruder 120, allowing the production of biomaterial 138 in a desired morphology (e.g., as a sheet or film, as described below with respect to methods 200 and 400). In at least some embodiments, extruder 120 may be configured to directly extrude material with a desired size (e.g., diameter), the extruded material being shaped into pellets or other suitable shape by a shaping device 136 integrated into (e.g., included in) extruder 120, eliminating the need for a separate shaping device 136.

When shaping device 136 is a pelletizer (e.g., as shown in FIG. 1) shaping device 136 may be configured to cut extruded material into a plurality of pellets or rods, or other shapes, to form a biomaterial 138. Biomaterial 138 may include agent 116 and polymer 110 that are homogenously mixed and that have solidified following extrusion through extruder 120. The content of agent(s) 116 in biomaterial 138 may substantially correspond to the ratio of agent 116 to polymer 110 introduced during step 304, and may be equivalent to any of the above-described concentrations or ranges of concentration. For example, a concentration of agent 116 may be within a range of about 0.5% by weight to about 30% by weight, within a range of about 1% by weight to about 20% by weight, or within a range of about 2% by weight to about 10% by weight. In particular, the concentration of agent 116 may be about 2% by weight, about 3% by weight, about 4% by weight, or about 20% by weight.

In at least some embodiments, biomaterial 138 may be formed with a desired concentration of agent 116 such that additional steps to dilute agent 116 are unnecessary. In these embodiments, biomaterial 138 may be formed with a concentration of agent 116 suitable for use with local drug delivery device, e.g., an implant. For example, a concentration of agent 116 within biomaterial 138 may be within a range of about 0.5% by weight to about 8% by weight, within a range of about 1% by weight to about 6% by weight, or within a range of about 2% by weight to about 4% by weight. In particular, a concentration of agent 116 within biomaterial 138 may be about 2% by weight, about 3% by weight, or about 4% by weight.

In other embodiments, biomaterial 138 may be formed with a relatively high concentration of agent 116 so as to form a stock batch of one or more immunosuppressive or neuro-regenerative agents integrated with polymer 110. The concentration of agent 116 in a stock batch of biomaterial 138 may be within a range of about 5% by weight to about 30% by weight, or about 8% by weight to about 20% by weight. In particular, the concentration of agent 116 in a stock batch of biomaterial 138 may be about 8% by weight, about 10% by weight, about 15% by weight, or about 20% by weight.

In examples where biomaterial 138 is intended for implantation after further processing during which a relatively high concentration of agent 116 is reduced to a desired level, the above-described process 300 may be repeated, for example by processing biomaterial 138 with the same or a different extruder 120, or one or more other devices, during which additional polymer (e.g., additional polymer 110) is added to biomaterial 138. This further performance of method 300 may facilitate the controlled reduction of the concentration of agent 116 within biomaterial 138. If desired, the further performance of process 300 may facilitate the incorporation of one or more second polymers that are different from polymer 110 used to produce biomaterial 138. Additionally or alternatively, process 200 and/or 400, described below, may enable the reduction of the concentration of agent 116 by the addition of additional polymer 110.

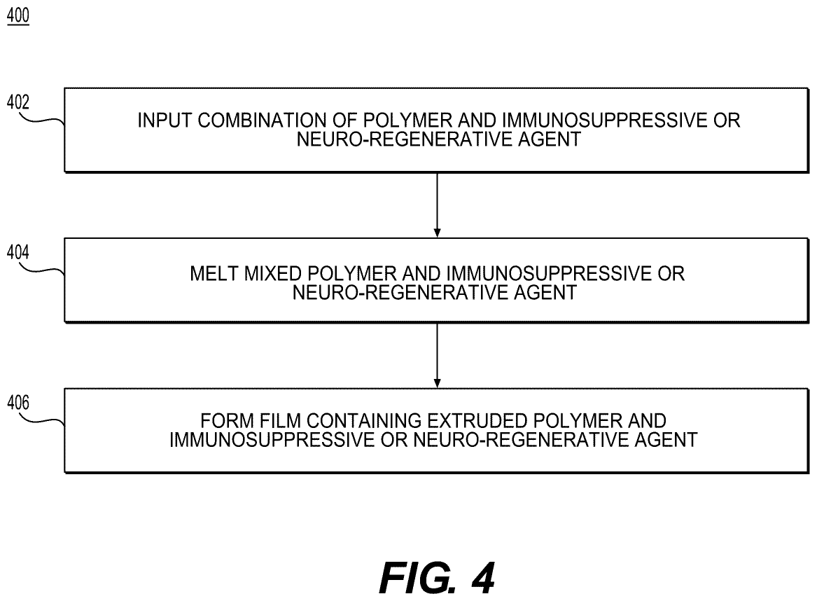

FIG. 4 illustrates a flowchart of an exemplary process 400 for producing a biomaterial that includes a polymer and one or more immunosuppressive or neuro-regenerative agents, such as FK506, rapamycin, or nimodipine, as a film. While process 400 is described in conjunction with process 200 and FIG. 2 below, as understood, process 400 may include fewer steps, additional steps, and/or different steps as compared to process 200. Additionally, process 400 may include fewer steps, additional steps, and/or different steps as compared to each block (e.g., steps 402, 404, and 406) illustrated in FIG. 4, or the specific order of the steps may be different. In some aspects, one or more steps may be repeated.

In a step 402 (FIG. 4), a mixture of polymer 110 and agent(s) 116 may be input into an extruder. The mixture may be obtained prior to performing step 402, or as part of step 402. A “mixture” may be a homogenous mixture or a heterogeneous mixture, including, for example, the polymer 110 and agent(s) 116 that were extruded in process 100 and/or process 300. While process 100 and/or process 300 provide examples that may be suitable for producing a mixture of polymer 110 and agent(s) 116 useful as an input material or mixture for processes 200 and 400, as understood, processes 100 and 300 are optional. The mixture of polymer 110 and agent(s) 116 used in processes 200 and 400 may be obtained by any suitable process, including processes that differ partially or entirely from processes 100 and 300.

Polymer 110 and agent(s) 116, whether produced in process 100 and process 300, pre-made, or produced by another method, may have a desired concentration of agent(s) 116 and may be suitable for use in an extrusion process for forming a film that is suitable for implantation in a human or non-human patient. As described above, the polymer 110 and agent(s) 116 may be present in biomaterial 138, in the form of particles such as spheres, pellets, or other forms.

The mixture of polymer 110 and agent(s) 116 may be input into an extruder 212, which may be configured to generate heat to melt polymer 110 and agent(s) 116 in step 404, described below. Extruder 212 may include a feeding device 214 to facilitate the introduction of the mixture to extruder 212, as well as an extruder screw 216 and a film die 218.

In some aspects, step 402 may include adding only biomaterial 138 to feeding device 214, which is desirable when biomaterial 138 contains a desired concentration of agent(s) 116 (e.g., 2%, 3%, or 4% of agent 116 such as FK506, rapamycin, or nimodipine). In configurations where biomaterial 138 is a stock batch containing an increased quantity of agent(s) 116, step 402 may include adding additional polymer 110, the additional polymer 110 being the same polymer (e.g., PDS) or one or more different polymers. The amount of additional polymer 110 may be precisely controlled by a device, as described above, to ensure that a desired ratio of biomaterial 138 to additional polymer 110 is introduced to extruder 212.

Feeding device 214 may be a hopper of an extruder 212, or may be a different type of feeding device, such as a vibratory feeder, similar to feeder 118 discussed above.

Step 404 may involve use of an extruder 212 that includes a single extruder screw 216 or plurality of extruder screws 216, having characteristics suitable for film extrusion. Extruder screw 216 may have threading with a consistent pitch as shown in FIG. 2, a continuously-varying pitch, or a pitch that forms multiple stages or zones (e.g., as described above with respect to extruder 120).

Regarding the characteristics of extruder screw 216, screw 216 may have a diameter of, for example, about 0.25 inch, about 0.5 inch, about 0.75 inch, about 1.0 inch, about 1.25 inch, about 1.5 inch, about 1.75 inch, or about 2.0 inch. Extruder screw 216 may be configured to generate torque of, for example, about 25 Nm, about 50 Nm, about 75 Nm, about 100 Nm, about 150 Nm, about 200 Nm, or about 250 Nm. Extruder screw 216 may be configured to operate (and generate) pressures of about 5,000 pounds per square inch (PSI), about 7,500 PSI, about 10,000 PSI, or about 15,000 PSI. In one example, extruder 212 may be a single-screw extruder with an extruder screw 216 having a 0.5 inch diameter, configured to generate 50 Nm of torque, and pressures of 10,000 PSI.

During step 404, extruder 212 may raise the temperature of polymer 110 and agent 116 (e.g., by use of heaters in addition to friction generated with screw 216 in some examples) to a desired temperature at or above the melting point of polymer 110. This desired temperature may depend on one or both of polymer 110 and agent(s) 116. Exemplary temperatures are described above with respect to step 306 of process 300. Extruder screw 216 may direct melted polymer 110 and agent(s) 116 to a distal end at which film die 218 is fluidly connected to extruder 212.

Step 406 may include forming a film that contains extruded polymer 110 and agent(s) 116. This may be performed with film die 218, for example. Film die 218 may include a distal opening 220 having dimensions suitable for forming a sheet with a desired width and thickness. In some aspects, die 218 may be configured to adjust the thickness of the extruded film, and may have a fixed width. The fixed width of opening 220 may be, for example, about 1.0 inch (25.4 mm), about 2.0 inch (50.8 mm), about 4.0 inch (101.6 mm or about 100 mm), about 6.0 inch (152.4 mm), or about 8.0 inch (203.2 mm). In some aspects, the width of opening 220 may be greater than about 2.0 inch or greater than about 4.0 inch.

If desired, die 218 may be provided with a deckle bar that facilitates the production of a film with consistent, smooth edges. A deckle bar of die 218 may regulate the rate at which material is extruded. Thus, a deckle bar of die 218 may define opposite lateral edges of the extruded film, such that these edges are uniform and substantially free of tearing or irregularities. In embodiments where a deckle bar is not present, a cutting device (not shown) may be connected downstream of extruder 212 and configured to remove material from one or both lateral edges of the material extruded from die 218.

The thickness defined by opening 220 may be adjustable from about 1 mm to about 60 mm, or from about 2 mm to about 40 mm, and may be configured to set a preliminary (e.g., non-final) or final thickness of the extruded film. Alternatively, both the width and thickness defined by slot-shaped opening 220 may be fixed, both the width and thickness may be adjustable, or the width may be adjustable.

The film extruded from opening 220 of die 218 may have a thickness that is larger than a desired thickness. This film may be provided, in a controlled manner, to a film take off unit 222 or other device that performs additional processing of the extruded film. Take off unit 222 may be configured to do one or more of cool (actively or passively), compress, and collect (e.g., via rolling) the film that exits die 218.

As shown in FIG. 2, take off unit 222 may include rollers 224 for drawing the film from die 218. Rollers 224 may pull the extruded film from die 218, while also stretching the film. The stretching of the film may decrease the thickness of the film compared to the thickness of the film exiting opening 220 of extruder 212. In some aspects, rollers 224 are rotated at a speed that results in a controllable translation speed of the film immediately downstream of rollers 224. Suitable speeds for the film, immediately downstream of rollers 224, include speeds of about 1 foot per minute (about 0.3 meters per minute) to about 20 feet per minute (about 6.1 meters per minute), about 5 feet per minute (about 1.5 meters per minute) to about 15 feet per minute (about 4.6 meters per minute), or about 7 feet per minute (about 2.1 meters per minute) to about 10 feet per minute (about 3.0 meters per minute). In some aspects, suitable speeds for the film, immediately downstream of rollers 224, may include speeds of about 3 feet per minute (about 0.9 meters per minute) to about 14 feet per minute (about 4.3 meters per minute), or about 5 feet per minute (about 1.5 meters per minute) to about 12 feet per minute (about 3.7 meters per minute).

Stretching may be accomplished by rotating rollers 224 at a speed that is greater than a speed at which material exits extruder 212. For example, film immediately downstream of rollers 224, corresponding, e.g., to one or more of the speed ranges for rollers 224 described in the preceding paragraph, may translate at a speed that is at least double (2X) the speed of film immediately downstream of opening 220. As another example, film immediately downstream of rollers 224 may translate at a speed that is at least triple (3X) the speed of film immediately downstream of opening 220. In particular, film immediately downstream of rollers 224 may translate at a speed that is at least quadruple (4X) the speed of film immediately downstream of opening 220. As another example, film immediately downstream of rollers 224 may translate at a speed that is at least five times (5X) the speed of film immediately downstream of opening 220. In some aspects, the speed of rollers 224 may be about two, about three, about four, or about five times the speed of the film as it exits opening 220 of extruder 212. In some aspects, the speed of rollers 224 may be about two to about five times the speed of the film as it exits opening 220 of extruder 212, may be about three to about five times the speed of the film as it exits opening 220 of extruder 212, may be about three to about four times the speed of the film as it exits opening 220 of extruder 212, or may be about two to about four times the speed of the film as it exits opening 220 of extruder 212. In some aspects, maintaining a relatively consistent speed of rollers 224 may promote consistency in the thickness of the film.

In some embodiments, take off unit 222 includes a pair of nip rollers. The nip rollers may form a space between them that is smaller than the thickness of the film material immediately prior to entering this space. This space, or gap G between the rollers, may be measured in a direction that is aligned with a radial direction of one or both of the rollers. Due to the size of gap G being smaller than the width of the material entering gap G, the nip rollers may compress or increase the consistency of the thickness of the material passing through the gap. In some embodiments, gap G is from about 5 μm to about 70 μm, from about 10 μm to about 60 μm, from about 20 μm to about 50 μm, or from about 30 μm to about 40 μm. In the configuration illustrated in FIG. 2, rollers 224 form nip rollers. In an alternate configuration, nip rollers can be positioned downstream of rollers 224 as a second or subsequent pair of rollers.

In some aspects, the size of the gap between rollers 224 may be substantially similar to the thickness of the film material immediately prior to entering gap G so that rollers 224 do not substantially compress the film material. In such an embodiment, rollers 224 may control the speed of the film, may increase the consistency of thickness of the film material passing through the gap, may smooth the film material passing through the gap, or may serve one or more other functions or combination of functions.

Take off unit 222 may include a coolant circulation system for supplying coolant to rollers 224, actively cooling rollers 224. Suitable coolants include, for example, water, ethylene glycol, and other fluids. One or both of these actively-cooled rollers 224 may be part of a coolant circuit 226 in which coolant is pumped to each roller (e.g., to a central portion of each roller 224), to maintain roller 224 and the material extruded from extruder 212 at or within a target temperature or target temperature range (collectively referred to as “target temperature” for ease of description). The temperature and flow of the coolant may be controlled via a compressor, heat exchanger, pump, or other known equipment.

In some embodiments, the target temperature, as measured by the temperature of coolant, is about 0 degrees Celsius to about 25 degrees Celsius. In particular, the target temperature of the coolant may be about 0 degrees Celsius, may be about 5 degrees Celsius, may be about 10 degrees Celsius, may be about 15 degrees Celsius, may be about 20 degrees Celsius, or may be about 25 degrees Celsius. In some embodiments, the target temperature may be measured at the rollers, or at the film itself, and may be about 0 degrees Celsius, may be about 5 degrees Celsius, may be about 10 degrees Celsius, may be about 15 degrees Celsius, may be about 20 degrees Celsius, or may be about 25 degrees Celsius.

In passively-cooled take off units, one or more fans, air condition systems, etc., may be used instead of coolant circuit 226. In some aspects, both passive and active coolant systems may be used. The coolant system may include one or more sensors to determine the temperature of coolant in real time. In a passive system, ambient temperature or the temperature of one or more rollers 224 may be measured. Based on the signals from these sensors, the coolant system may monitor and control the target temperature automatically and/or display a current temperature to facilitate manual observation and control. Cooling of rollers 224 may reduce the tackiness of the film to inhibit the from sticking to the machinery of take off unit 222.

If desired, a release liner 228 may be introduced downstream of opening 220. For example, release liner 228 may be introduced at rollers 224, with release liner 228 sandwiching the film extruded from 218 and preventing the film from contacting the material (e.g., metal) of rollers 224. Release liner 228 may be introduced at substantially the same speed as the rotation of rollers 224. Release liner 228 may include a silicone or other non-stick material that is configured to contact the film for an extended period of time and be subsequently removed without damaging the film.

Wound film 232 may be collected on a collection roller 230, for example. Film 232 may have a desired thickness. In some aspects, the thickness of film 232 may be at least partially controlled (e.g., by compression) with nip rollers that are separate from take off unit 222 or nip rollers integrated into pulling rollers 224 of take off unit 222, as shown in FIG. 2 and described above. In other aspects, take off unit 222 may include both nip rollers and rollers 224. In other aspects, the rollers may not substantially affect the thickness of film 232.

Film 232 may have a thickness of about 10 μm to about 200 μm, 30 μm to about 120 μm, e.g., about 30 μm to about 60 μm, about 50 μm to about 110 μm (or about 60 μm to about 90 μm), or about 90 μm to about 120 μm. In some aspects, step 406 may include setting, via unit 222 or another device, a desired thickness for a particular application. In some aspects, properties of film 232 may be favorably controlled by selecting a desired thickness. These controllable properties may include handling, initial release of agent 116 (e.g., burst release), a duration of time during which a therapeutic dose of agent 116 is delivered, a total amount of agent 116 delivered, and an average (e.g., daily) amount of agent 116 delivered.

If desired, the width of film 232 may also be adjusted via unit 222 (e.g., by using a die formed with a wider lip, a narrower lip or an adjustable lip) or another device, such as a separate cutter. This may allow the width of film 232, in addition to the thickness of film 232, to be controlled, and if desired, reduced. Taking the example of a film 232 extruded via opening 220 with an approximately 2.0 inch (50.8 mm) width, take off unit 222 may reduce the width of film 232 to about 1.5 inch (38.1 mm), about 1.0 inch (25.4 mm), about 0.75 inch (19.1 mm), about 0.5 inch (12.7 mm), or about 0.25 inch (6.4 mm). In a particular example, film 232 may have a width of about 0.67 inch (17 mm) following processing with unit 222. These widths may be present in each configuration of film 232 described below.

In some aspects, the width of film 232 may be achieved by compressing and/or stretching film 232 with take off unit 222 or another device, such that film 224 collected on collection roller 230 has a final desired width. If desired, the width of film 232 on 230 may be larger than desired, and may be cut when ready for use, for shipping, or for storage. The length of film 232 may also be modified by cutting the collected film 232 prior to use, prior to shipping, or prior to storage.

Film 232, once formed as described with respect to polymer 110 and agent 116 in step 406, may be suitable for use without additional processing, except for cutting and shaping. Alternatively, film 232 may be sterilized (e.g., via gamma irradiation) or otherwise treated prior to use. To facilitate use as or with an implant, film 232 may be further processed to add one or more attachment points (e.g., suture holes and/or surrounding features to increase visibility of the holes and/or reinforce the suture point). These attachment points may be formed in the manner described in U.S. Pat. No. 11,166,800, filed on Apr. 11, 2019, which issued on Nov. 9, 2021, the entirety of which is incorporated by reference.

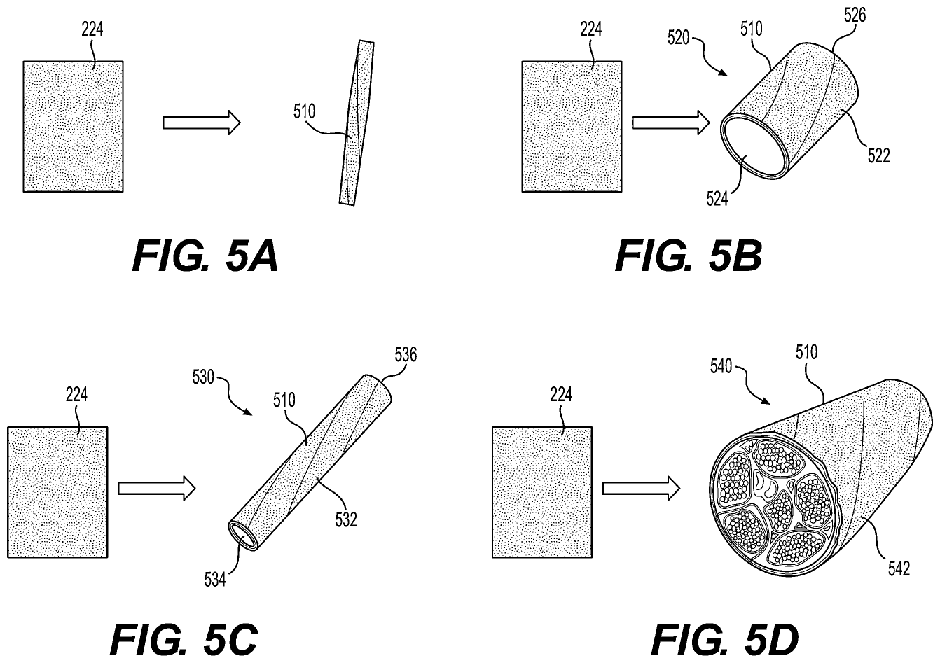

FIGS. 5A-5D illustrate various form factors that may be realized by film 224 alone, or by combining film 224 with an implant (e.g., a nerve implant) to form an implant assembly or biomaterial assembly. In a first example illustrated in FIG. 5A, film 224 may be suitable for implantation. Film 224 may be located at an injury or other surgical site and may be formed into a generally tubular wrap 510 that surrounds nerve tissue (not shown). This wrap 510 may be relatively easy to suture, may have structures that retain sutures (e.g., pre-formed attachment points and/or attachment points formed during use), and may be transparent to facilitate visualization of the nerve tissue around which wrap 510 is wrapped, although it may not be transparent in yet other embodiments. Wrap 510 may be in the form of a sheet, the sheet having been cut from a larger film 224. In embodiments where film 224 is used as a wrap 510 with one or more additional implant structures, wrap 510 may be secured to the other implant structures via friction (i.e., without the use of adhesive), with one or more layers of adhesive, and/or by sutures or other mechanical fasteners suitable for use in a subject. In some aspects, wraps formed of other materials may be layered with wrap 510, e.g., with alternating layers of other materials and wrap 510, with wrap 510 sandwiched between layers of other materials, or with wrap 510 as one or more outer layers of a multi-layer wrap including other materials.

Other materials that wrap 510 may be used in conjunction with include, e.g., for example, porcine small intestine submucosa (“SIS”), amnion-based tissue (e.g., amniotic/chorionic membrane), or reconstituted denatured collagen. If desired, materials used with wrap 510 may include one or more synthetic materials instead of or in addition to a natural material, such as SIS. Suitable synthetic materials may include a reabsorbable polymer formed as one or more layers in a non-woven or woven structure, include homopolymers, copolymers, and/or polymeric blends of one or more of the following monomers: glycolide, lactide, caprolactone, dioxanone, trimethylene carbonate, monomers of cellulose derivatives, or monomers that polymerize to form polyesters. Additional synthetic materials that may be suitable for use with wrap 510 instead of, or in addition to a natural material, include silicone membranes, expanded polytetrafluoroethylene (cPTFE), polyethylene tetraphthlate (Dacron), polyurethane aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof. Natural polymers suitable for use with wrap 510 may include collagen, elastin, thrombin, fibronectin, starches, poly(amino acid), gelatin, alginate, pectin, fibrin, oxidized cellulose, chitin, chitosan, tropoclastin, hyaluronic acid, fibrin-based materials, collagen-based materials, hyaluronic acid-based materials, glycoprotein-based materials, cellulose-based materials, silks and combinations thereof.

Wrap 510 may have suitable handling properties, and may be configured to be draped around an injured nerve or other tissue, and may be resistant to kinking, tearing, or bunching. The thickness (measured in a direction perpendicular to a plane defined by wrap 510), length (measured along a direction in which wrap 510 was extruded), and/or width (measured perpendicular to the thickness and length) of film 224, and thus the size of wrap 510, may be set prior to use. Alternatively, the thickness and width of film 224 may be set as described above (e.g., film 224 may be about 30-200 μm thick, about 30-120 μm thick, about 30-60 μm thick, about 50-110 μm thick, about 60-90 μm thick, or about 90-120 μm thick) prior to implantation, and the length may be determined by trimming film 224 before wrapping film 224 to define wrap 510. In some procedures and/or embodiments, the length of film 224 may also be trimmed prior to implantation. In some aspects, wrap 510 may be sold in different sizes to accommodate different use cases.

In some aspects, wrap 510 may be configured to be implanted at the site of a peripheral nerve injury in a subject. Wrap 510 may be formed by rolling a substantially rectangular sheet or film 224 (as shown in FIG. 5A), a circular film, or a film having other regular or irregular shapes. While one film 224 is shown in FIG. 5A, wrap 510 may include a plurality of layers of sheets or films stacked and secured together. In some aspects, proximal and distal ends of wrap 510 may be suitable for being secured (e.g., with sutures, adhesives, or other mechanisms) to soft tissue so as to form a barrier that protects nerve tissue during healing.

FIG. 5B illustrates a biomaterial film 224 used as a wrap 510 around a tubular nerve wrap implant 522 (also referred to as a nerve connector implant), forming an implant assembly 520. While FIG. 5B depicts wrap 510 located on an outer surface of implant 522, in some aspects, wrap 510 may be used to cover an inner surface of implant 522, or both an inner and outer surface. While FIG. 5B, as well as FIGS. 5C and 5D described below, shows a plurality of wraps 510 layered on a particular implant (e.g., nerve wrap implant 522), as understood, a single wrap 510 may be present on or within implant 522. Additionally, one or more wraps 510 may overlap, may form gaps where no wrap 510 is present, may form ends of implant 522 where no wrap 510 is present, etc. As understood, similar modifications are also possible for the implant assemblies 530 and 540 below.

In some aspects, implant assembly 520 may be rectangular (e.g., provided as a sheet or plurality of sheets, or otherwise unwrapped), having a length measured from proximal end 524 to distal end 526 that is longer than a width of implant assembly 520 measured in a direction perpendicular to the length. In particular, implant assembly 520 may have a length, as measured from proximal end 524 to distal end 526, within a range of about 5 mm to about 60 mm, within a range of about 10 mm to about 50 mm, or within a range of about 20 mm to about 40 mm. In particular, a cylindrical body formed when implant assembly 520 is rolled (or prior to rolling) may have a length of about 20 mm or about 40 mm.

When implant assembly 520 is in a cylindrical (e.g., rolled or wrapped) form, implant assembly may define a tubular body with a diameter within a range of about 0.5 mm to about 10 mm, about 1.0 mm to about 8 mm, or about 1.5 mm to about 7 mm. In particular, a diameter of the body of assembly 520 may be equal to about 1.5 mm, about 2.0 mm, about 3.0 mm, about 4.0 mm, about 5.0 mm, about 6.0 mm, or about 7.0 mm. A length of the body of implant assembly 520 may be within a range of about 5 mm to about 20 mm, or within a range of 10 mm to about 15 mm. In particular, a length of cylindrical body 424 may be equal to about 10 mm or equal to about 15 mm.

Films or wraps 510 of implant assembly 520 may be provided at different locations throughout implant 522. In some aspects, a higher concentration of agent 116 may be provided by placing wraps 510 at a location of implant 522 that is expected to be positioned adjacent to an injured nerve, such as a nerve end. In such cases, a higher concentration of agent(s) 116, such as FK506, may be provided at proximal end 524, at distal end 526, and/or at an axial center portion including the halfway-point between ends 524 and 526. Alternatively, wraps 510 may be substantially consistently and regularly distributed throughout the entirety of implant 522. Additionally, while biomaterial wrap 510 may include a neuro-regenerative or immunosuppressive agent, wrap 510 may include one or more growth-inhibiting agents, e.g., that may prevent or reduce the formation of a neuroma.

FIG. 5C illustrates an exemplary implant assembly 530 that may be suitable for implantation in a subject. In some aspects, implant assembly 530 may extend from a proximal end 534 to distal end 536 to define a rod or cylinder with a hollow interior (like implant assembly 520) that may be useful as a nerve protector. Implant assembly 530 may be a pre-rolled implant 532 for case of use, if desired. Implant assembly 530 may be configured to be implanted at the site of a peripheral nerve injury in a subject. In particular, implant assembly 530 may be configured for attachment at a peripheral nerve injury site to provide a structural barrier for protecting one or more peripheral nerves or nerve ends, as well as structural reinforcement to support peripheral nerve reconstruction and healing. Implant assembly 530 may include an implant 532 with a cylindrical body formed in the shape of a rod or tube, with one or more wraps 510 attached to an outer circumferential surface of implant 532 (as shown in FIG. 5C) or to an inner surface of implant 532.

In some aspects, implant assembly 530 may have a length that is longer than a length of implant assembly 520. In particular, the cylindrical body of implant assembly 530 may have a length, as measured from end 534 to end 536, within a range of about 5 mm to about 60 mm, within a range of about 10 mm to about 50 mm, or within a range of about 20 mm to about 40 mm. In particular, implant assembly 530 may have a length of about 20 mm or about 40 mm. Implant assembly 530 may have a diameter within a range of about 1.0 mm to about 20 mm, about 1.5 mm to about 15 mm, or about 2 mm to about 10 mm. In particular, a diameter of the cylindrical body of assembly 530 may be about 2 mm, about 3.5 mm, about 5 mm, about 7 mm, or about 10 mm.

Biomaterials wraps 510, when attached to or otherwise incorporated with implant 532, may be provided at different locations throughout the cylindrical body of implant 532. In some aspects, a higher concentration of agent 116 may be provided at distal end 534 and proximal end 536 as compared to an axial center portion of implant 532, or vice versa. Alternatively, wraps 510 may be placed on implant 532 such that agent(s) 116 are substantially consistently and regularly distributed throughout the entirety of implant 532, including along its length.

FIG. 5D illustrates an exemplary implant assembly 540 that may be suitable for implantation in a subject. In some aspects, implant assembly 540 may include a nerve graft implant 542 formed of decellularized material that may be implanted at the site of a peripheral nerve injury in a subject. While implant assemblies 520 and 530 may each have a hollow interior, implant assembly 540 may have a partially or entirely solid interior. For example, implant 542 of assembly 540 may include decellularized epineurium, decellularized perineurium, and/or decellularized endoneurium in an interior thereof. One or more biomaterial films 224 may be placed and attached to an outer circumference of implant 542, forming an exterior surface that is at least partially formed by biomaterial wraps 510, as shown in FIG. 5D.

Biomaterial wraps 510 of implant assembly 540 may generally extend along a length of assembly 540, as shown in FIG. 5D. Similar to implant assemblies 520 and 530, biomaterial wraps 510 may supply a relatively higher concentration at one or more locations, such as proximal and distal ends of implant 542. In some aspects, proximal and/or distal ends of implant 542 may be exposed, and thus substantially free of biomaterial wraps 510 and agent 116 at the time of assembly and/or during initial implantation, if desired. Implant 522 and implant 532 may similarly have ends free of wraps 510, if desired.

Each of the above-described embodiments, including wrap 510 and implant assemblies 520, 530, and 540, may be configured to deliver agent(s) 116 in a localized, sustained, and controlled manner. For example, these biomaterials assemblies and implants may enable accurate loading of an active ingredient, such as one or more neuro-regenerative or immunosuppressive agents (e.g., FK506, rapamycin, or nimodipine), into a matrix of a polymer to enable controlled release of the agent(s) when incorporated with other devices or implants.

In particular, incorporation of FK506 within a polymeric delivery system formed with one or more of the above-described biomaterials may allow localized release of FK506 while axons regenerate toward target end tissue or organs. Local delivery of one or more neuro-regenerative or immunosuppressive agents may be desirable to increase the number of neurons that are able to regenerate their axons, as well as increase the rate of axonal regeneration. The production of a biomaterial containing FK506 may be useful as a universal or modular delivery system that enables the formation of bioactive-releasing implants and devices, e.g., FK506-releasing implants and devices, in a plurality of different form factors that are useful in different types of injuries and, in particular, different types of peripheral nerve injuries. The biomaterials described above may also be useful for incorporating an active ingredient, such as one or more neuro-regenerative or immunosuppressive agents, without requiring the addition of these agent(s) after the delivery device (e.g., an implant) has been formed. These biomaterials may be useful with one or more neuro-regenerative or immunosuppressive agents, such as FK506, that have a molecular weight less than about 1,200 g/mol, or less than about 1,000 g/mol. The above-described biomaterials may also be useful with hydrophobic neuro-regenerative or immunosuppressive agents, such as FK506.

At least some of the above-described advantages associated with use of FK506 as agent 116 may be realized by using rapamycin as agent 116. For example, rapamycin may facilitate axonal regeneration and provide neuroprotective functions.

Additionally, at least some of the above-described advantages associated with the use of FK506 as agent 116 may be realized by using nimodipine as agent 116. For example, nimodipine may facilitate axonal regeneration and generation of myelin.

### EXAMPLES

The disclosure may be further understood by the following non-limiting examples. The examples are intended to illustrate embodiments of the above disclosure, and should not be construed as to narrow its scope. While these examples involve the use of FK506 and PDS, as understood, the disclosed embodiments are not limited to this particular agent and polymer. One skilled in the art will readily recognize that the examples suggest many other ways in which the embodiments of the disclosure could be practiced. It should be understood that numerous variations and modifications may be made while remaining within the scope of the disclosure.

### Example 1, Part A: FK506-Incorporated in Films of Polydioxanone (PDS) Having Different Thicknesses

Biomaterial films were prepared using a two-stage process, including a first stage in which PDS pellets containing 2% FK506 were first formed, and then used, in a second stage, to prepare a biomaterial film having a target thickness. Biomaterial films with three different target thicknesses were produced, each with a 2% concentration of FK506 by weight, including a first target thickness of 30-60 μm, a second target thickness of 50-110 μm, and a third target thickness of 90-120 μm.

To prepare the stock batch of pellets, PDS flakes were chilled using liquid Nitrogen. The chilled PDS flakes were then milled using a centrifugal mill to form powdered PDS. Residual moisture was removed from the powdered PDS by using a polymer dryer overnight.

FK506 was then mixed with the dried PDS powder using a speed mixer. This mixture of FK506 and PDS contained 2% FK506 by weight. The mixture was collected and introduced into a hopper of a twin-screw extruder by a vibratory feeder. The extruder included a pair of 18 mm diameter screws, each formed with metering, kneading, and shearing sections. Rotation of the pair of screws in the extruder heated the FK506 and PDS mixture to a temperature that reached a maximum of 135 degrees Celsius by generating torque of no more than 71 Nm.

The extruded FK506 and PDS was received, in rod form, by a pelletizer. The pelletizer broke the extruded rods into a plurality of pellets (i.e., shortened rods) using rotating knives configured for speeds of up to 24 meters per minute.

Films were then prepared using the PDS pellets containing 2% FK506. Pellets were introduced to a hopper of a single-screw extruder having a 0.5 inch diameter. Heat generated by the extruder melted the pellets, after which the extrudate was supplied to a 2 inch die with an adjustable lip, the die forming a film. The film was collected by a film take off unit, which drew the film on a chill roll and compressed the film to the target thickness.

### Example 1, Part B: FK506 Release Analysis from PDS Films

Biomaterial samples were prepared according to Example 1, Part A, including a first group of six film samples having a target thickness of 30-60 μm, a second group of six film samples having a target thickness of 50-110 μm, and a third group of six film samples having a target thickness of 90-120 μm. Each group contained 2% FK506 by weight. Each of the three groups of samples was evaluated to determine release kinetics of FK506 from the biomaterial films.

To prepare the three groups, individual films were separated from the roll and placed into a vial containing saline buffer solution. Each biomaterial-containing vial was placed in a bath having a temperature maintained at 37 degrees Celsius, to mimic human body temperature. While maintaining the temperature of each biomaterial at about 37 degrees Celsius, the saline buffer was removed from each vial at various time points and analyzed for FK506 content using liquid chromatography tandem mass spectrometry (LC-MS/MS). After collection at each time point, the saline buffer from each vial was replaced with fresh saline buffer.

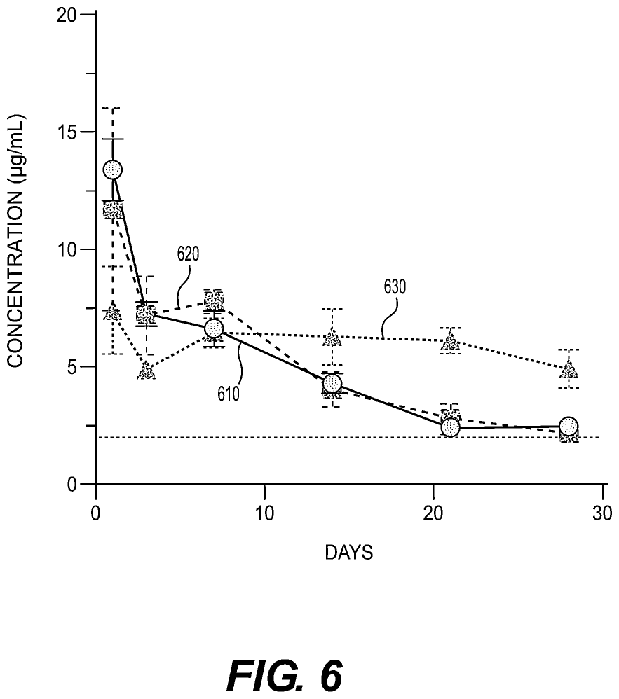

Collected samples of buffer solution from each vial were analyzed by LC-MS/MS to determine the amount of FK506 released from fibers at 1 day, 3 days, 7 days, 14 days, 21 days, and 28 days. The resulting concentrations of FK506 released from the fibers is represented in FIG. 6. In FIG. 6, each plotted point (i.e., circles, squares, and rectangles) represents the mean concentration of FK506 in the collected saline buffer for measurements of six samples, each sample having an initial concentration of 2% FK506 by weight. Each plotted circle represents the mean concentration of FK506 in the collected saline buffer for measurements 610 of samples having a thickness of 30-60 μm. Each plotted square represents the mean concentration of FK506 in the collected saline buffer for measurements 620 of the six samples having a thickness of 50-110 μm. Each plotted triangle represents the mean concentration of FK506 in the collected saline buffer for measurements 630 of the six samples having a thickness of 90-120 μm.

In some aspects, it may be desirable to achieve a release of one or more neuro-regenerative or immunosuppressive agents, such as FK506, for a particular period of time. In particular, it may be desirable to achieve release of FK506 for a period of at least 7 days, at least 14 days, or at least 28 days, the release of FK506 during this period being sufficient to ensure that the effective concentration of FK506, and/or other agent, remains within a therapeutic window. Additionally, it may be desirable to avoid burst release of the one or more neuro-regenerative or immunosuppressive agents, such as FK506. Avoiding a burst release may be desirable, for example, to enable a longer overall duration of FK506 release and/or to avoid the likelihood of a local concentration exceeding an upper limit of a therapeutic window.

As can be seen in FIG. 6, the effective concentration of FK506 remained in a therapeutic window of at or above about 2.1 ug/mL and well below a toxic dose of about 5 mg/ml in each measured sample over the 28-day period measurements were taken. In particular, for each of the three groups of films, the mean concentration of FK506 was observed between about 2.1 ug/mL and about 20 ug/mL for the full 28-day period. Additionally, no significant burst release was observed.

### Example 2: In Vitro Analysis of Nerve Regeneration Enhancement in Presence of FK506-Containing Films

Eight biomaterial films were prepared according to Example 1 parts A and B. The biomaterial films each contained 2% by weight FK506 and had a thickness of 30-60 μm. Blank films containing solely PDS and no FK506 (and no other neuro-regenerative agent or immunosuppressive agent) were produced for comparison.

Four treatment groups were prepared, each group having eight samples. In each group, a cluster of dorsal root ganglion cells were treated to obtain dissociated sensory neurons. These dissociated neurons were placed in a well containing culture media, the well having been subsequently covered with a laminin-coated cover slip. The cells were cultured at 37 degrees Celsius for a period of 48 hours. After culturing, cover slips were stained and visual analysis of the stained sensory neuron cells was performed to evaluate the presence of extended neurites.

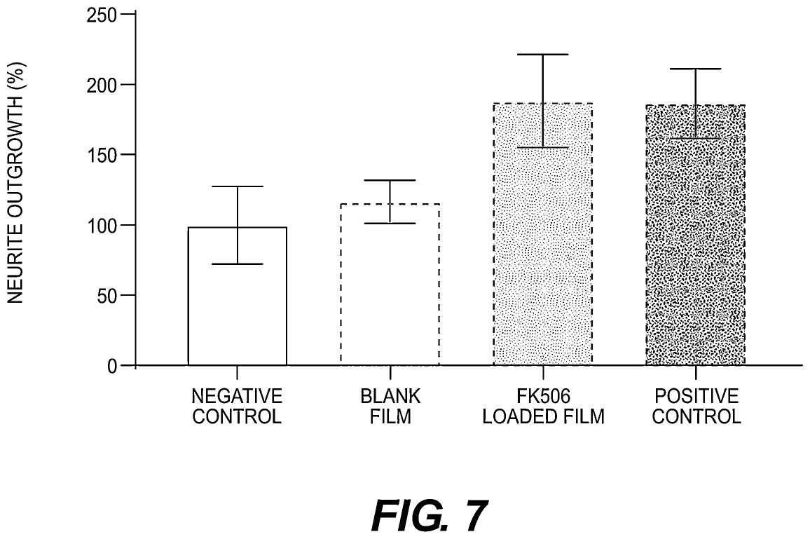

FIG. 7 is a bar chart visualizing neurite outgrowth that was observed. Values shown in FIG. 7 are normalized based on the below-described negative control by setting the observed neurite outgrowth as the value “100”. Thus, a result of “150” represents 50% greater observed neurite outgrowth as compared to the negative control.

In FIG. 7, the negative control corresponds to sensory neurons that were cultured as described above, without the presence of a film and in the absence of FK506. As described above, the blank film results correspond to sensory neurons that were cultured in the presence of a PDS film having a thickness of 30-60 μm and containing no FK506. The FK506 loaded film corresponds to sensory neurons that were cultured in the presence of biomaterial films containing 2% by weight FK506 that had a thickness of 30-60 μm. The positive control corresponds to sensory neurons that were cultured in the presence of FK506 solution and no film.

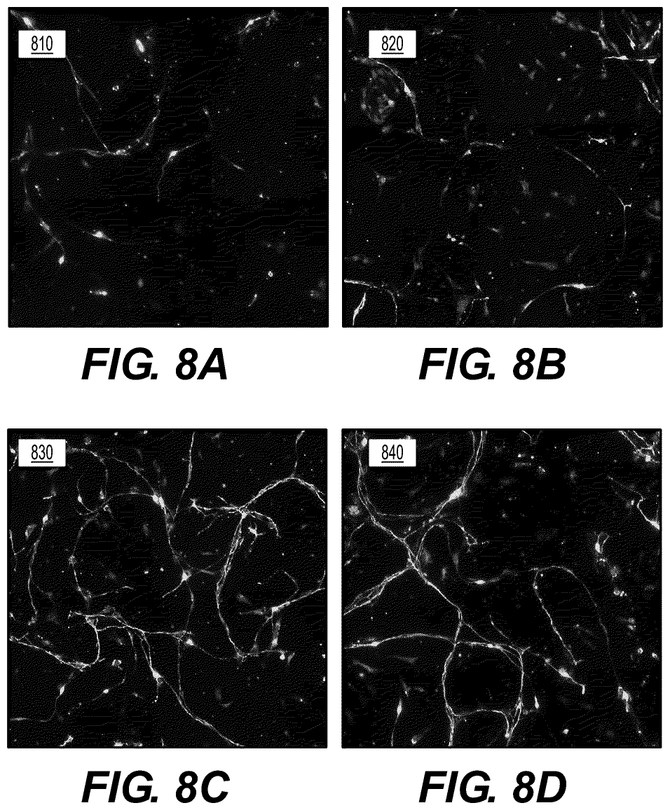

FIGS. 8A-8D provide representative images of each of the four treatment groups described above. In FIGS. 8A-8D, light colors indicate the presence of neurite extension, which correlates to nerve regeneration capacity. Dark portions indicate the absence of neurite extension. Image 810 in FIG. 8A corresponds to the negative control, image 820 in FIG. 8B corresponds to the blank film, image 830 in FIG. 8C corresponds to the FK506 loaded film (e.g., as prepared according to Example 1, parts A and 1B), and image 840 in FIG. 8D corresponds to the positive control.

As can be seen in FIGS. 7 and 8A-8D, neurite extension was significantly increased by the presence of FK506. No statistically-significant difference was observed between the FK506 loaded film and the use of FK506-containing solution. This indicates the suitability of FK506-containing film for in vivo use where FK506-containing solution may be unsuitable or impractical (e.g., due to the inability to control migration of the solution).

It should be understood that although the present disclosure has been made with reference to preferred embodiments, exemplary embodiments, and optional features, modifications and variations of the concepts herein disclosed may be resorted to by those skilled in the art, and that such modifications and variations are considered to be within the scope of this disclosure as defined by the appended claims. The specific embodiments and examples provided herein are examples of useful embodiments of the present disclosure and are non-limiting and illustrative only. It will be apparent to one skilled in the art that the present disclosure may be carried out using a large number of variations of the devices, device components, methods, and steps set forth in the present description. As will be recognized by one of skill in the art, methods and devices useful for the present methods can include a large number of various optional compositions and processing elements and steps.

What is claimed is:

1. A method of preparing an implantable biomaterial film, comprising:
inputting a combination of a polymer and a neuro-regenerative agent or an immunosuppressive agent into an extruder;
melting the polymer within the extruder; and
extruding the combined polymer and the neuro-regenerative agent or the immunosuppressive agent to form the implantable biomaterial film.

2. The method of claim 1, wherein the neuro-regenerative agent or the immunosuppressive agent includes FK506, rapamycin, or nimodipine.

3. The method of claim 1, wherein the implantable biomaterial film has a thickness of about 10 μm to about 200 μm.

4. The method of claim 1, wherein the implantable biomaterial film has a width of about 100 mm to about 6.4 mm.

5. The method of claim 1, further comprising incorporating the implantable biomaterial film as part of an implant.

6. The method of claim 1, further comprising incorporating the implantable biomaterial film into one of a nerve connector, a pre-rolled nerve wrap, a sheet-like nerve wrap, or a nerve graft.

7. The method of claim 5, wherein the implant is a multi-layer implant.

8. The method of claim 5, wherein the implantable biomaterial film is attached to an inner surface of the implant.

9. The method of claim 5, wherein the implantable biomaterial film is attached to an outer surface of the implant.

10. The method of claim 5, wherein a layer of the implant comprises a synthetic material in addition to the film.

11. The method of claim 1, wherein the neuro-regenerative agent or the immunosuppressive agent comprises FK506, and the combination of the polymer and the FK506 input into the extruder includes about 1% to about 20% FK506, as measured by weight.

12. The method of claim 1, wherein the neuro-regenerative agent or the immunosuppressive agent includes a plurality of neuro-regenerative agents, a plurality of immunosuppressive agents, or at least one neuro-regenerative agent and at least one immunosuppressive agent.

13. The method of claim 1, wherein the neuro-regenerative agent or the immunosuppressive agent including at least one agent that is both neuro-regenerative and immunosuppressive.

14. The method of claim 1, further comprising reducing a thickness of the extruded implantable biomaterial film by compressing and/or stretching the implantable biomaterial film using a film take off unit.

15. The method of claim 14, wherein the film take off unit includes a pair of nip rollers that are spaced apart by a gap of about 10 μm to about 60 μm.

16. The method of claim 14, further comprising operating the film take off unit at an approximately constant speed to facilitate formation of the implantable biomaterial film with an approximately constant thickness.

17. The method of claim 14, further comprising operating the film take off unit at a speed that is about 2 times to about 5 times faster than a speed at which the polymer is extruded.

18. The method of claim 14, wherein the film take off unit is operated at a speed such that a speed of the film immediately downstream of a roller of the film take off unit is about 0.3 meters per minute to about 6.1 meters per minute.

19. The method of claim 14, further comprising collecting the implantable biomaterial film with the film take off unit.

20. The method of claim 14, wherein a roller of the film take off unit is cooled with a liquid coolant.

21. The method of claim 1, further comprising cutting the implantable biomaterial film into a plurality of sheets.

22. The method of claim 1, wherein the polymer is a homopolymer, copolymer, and/or polymeric blend including one or more of the following monomers: glycolide, lactide, caprolactone, dioxanone, trimethylene carbonate, monomers of cellulose derivatives, and monomers that polymerize to form polyesters.

23. The method of claim 1, wherein the polymer comprises polydioxanone.

24. The method of claim 1, wherein the polymer comprises polydioxanone and a second polymer copolymerized with the polydioxanone.

25. The method of claim 1, wherein the polymer comprises polydioxanone (PDS) and poly(trimethylene carbonate), poly(glycolide), poly(d,l-lactide), poly(l-lactide), or poly(caprolactone) as a copolymer with the PDS.

26. The method of claim 25, wherein the copolymer is a random copolymer, the polymer comprising about 40% to about 90% PDS, by molecular weight.

27. The method of claim 25, wherein the copolymer is a block copolymer, the polymer comprising about 45% to about 85% PDS, by molecular weight.

28. The method of claim 1, wherein the polymer comprises polydioxanone (PDS) and at least one of poly(glycolide), poly(l-lactide), or poly(d,l-lactide) as a copolymer with the PDS.

29. The method of claim 23, wherein the polymer is surface treated with polyethylene glycol.

30. The method of claim 1, wherein the polymer contains a basic salt, the basic salt being about 0.5% to about 10% of the polymer, by molecular weight.

31. The method of claim 1, further comprising cooling the implantable biomaterial film and collecting the implantable biomaterial film, the implantable biomaterial film including the polymer combined with FK506 as the neuro-regenerative or immunosuppressive agent.
